(19) United States
(12) Patent Application Publication
(10) Pub. No.: US 2003/0224038 A1
(43) Pub. Date: Dec. 4, 2003

(54) Cross-linkable phosphonate-containing supramolecular complexes and their use for delivery of therapeutic and diagnostic agents

(76) Inventors: Catherine J. Page, Eugene, OR (US); Marcus Helfrich, State College, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/370,221

(22) Filed: Feb. 20, 2003

Related U.S. Application Data
(60) Provisional application No. 60/359,100, filed on Feb. 21, 2002.

(51) Int. Cl. A61K 38/14; A61K 31/704; A61K 31/7048; A61K 31/525; A61K 31/519; A61K 31/517; A61K 31/4745; A61K 31/337; A61K 31/407; A61K 9/127; A61K 33/24

(52) U.S. Cl. 424/450; 514/27; 514/34; 514/283; 514/410; 514/449; 514/8; 514/251; 424/649; 514/264.1; 514/266.1

(57) ABSTRACT

Cross-linkable, phosphonate containing supramoleuclar aggregates are disclosed, which may be used to advantage, for example, as drug delivery vehicles for the treatment of bone-related disorders.

### CROSS REFERENCES TO RELATED APPLICATIONS

This application claims priority under 35 U.S.C. §119(e) to U.S. Provisional Patent Application No. 60/359,100 filed on Feb. 21, 2002, the entire disclosure of which is incorporated by reference herein.

### GOVERNMENT RIGHTS

Pursuant to 35 U.S.C. §202(c) it is acknowledged that the U.S. Government has certain rights in the invention described herein, which was made with funds from the National Science Foundation, Grant Number NSF CHE-9633062.

### FIELD OF THE INVENTION

This invention relates to the field of delivery of therapeutic and diagnostic agents in vivo. Specifically, novel compositions are provided comprising phosphonate-containing supramolecular aggregates which may be used to advantage as delivery vehicles for therapeutic and diagnostic agents useful in the treatment and/or diagnosis of a variety of pathological disorders.

### BACKGROUND OF THE INVENTION

Several publications and patent documents are referenced in this application in order to more fully describe the state of the art to which this invention pertains. The disclosure of each of these publications is incorporated by reference herein. A numerical listing of the referenced publications is provided at the end of this specification.

Rapid advances in the field of rational drug delivery have yielded a plethora of new and more efficient means for treating and diagnosing a wide range of pathological conditions, including various types of cancer (1-3). However, these therapeutic or diagnostic agents are often introduced into the body through the bloodstream, and only a small fraction of the active agent actually reaches the affected area (1,4). The remaining quantities are taken up by other body tissues where they can have toxic side effects, or are scavenged from the bloodstream as waste. As a result, there is an increased need for strategic methods to target these agents directly to the disease site to increase their efficiency. Over the past two decades, methods for either incorporating therapeutic or diagnostic agents into vesicles (1, 4-6) or polymer micelles (6-9), or attaching them directly to molecular carrier structures that can serve as transporters have been investigated. Such carrier molecules can be specifically functionalized to target delivery of therapeutic or diagnostic agents to a specific organ or region of interest, thus reducing the amount of agent lost to other cells (10).

Micelles are formed when amphiphilic molecules are dissolved in water above some critical concentration (the critical micelle concentration, or CMC) (11-13). Amphiphilic molecules are molecules which have polar and nonpolar regions, such as a soap molecule which has a polar (usually charged) head group and a long hydrophobic hydrocarbon ‘tail’. In an aqueous environment, the amphiphilic molecules self-aggregate to form structures in which the hydrophobic portions of the molecules are on the interior of the structure, and the hydrophilic polar head-groups are exposed to the aqueous solution. When the amphiphiles form double-layer structures (akin to cell membranes), these structures are known as vesicles. Micelles are dynamic structures that are in equilibrium with the monomers from which they are composed.

Because of their dynamic nature, micelles have been previously studied as potential drug delivery systems (6, 7, 9). Most of this prior research has focused on the use of block copolymers (which form micelles at low concentrations that are exceptionally stable) as vehicles for delivering anti-cancer drugs in vivo (7). These polymers are often composed of a hydrophobic chain of a specific length of a polyamino acid. A hydrophobic drug molecule can then be covalently linked to the amino acid backbone. Upon dialysis from an organic phase, the polymer chains quickly associate to form micelles to protect the hydrophobic amino acid-drug segment of the molecules. When the polymer-drug matrix is introduced in vivo, the drug can be cleaved from the amino acid backbone through hydrolysis and can then diffuse into the body.

Perhaps the biggest shortcoming of the systems described above is their inability to deliver the encapsulated drug to specific areas of the body. The use of antibodies and proteins as a means of targeting does not prevent the delivery vehicle from binding to receptor sites located in tissues other than those targeted by the antibody or protein. Also, reaction with serum proteins and other biocomponents could lead to the aggregation of the solubilized material within the bloodstream. As a result, such functionalization may lead to toxic side effects that limit the utility of these materials, especially for chemotherapeutic use.

### SUMMARY OF THE INVENTION

In accordance with one aspect of the present invention, there is provided a composition which comprises a therapeutic or diagnostic agent and stabilized supramolecular aggregates comprising a phosphonate or a combination of chemically distinct phosphonates, the phosphonate (s) having hydrophillic head groups, and hydrophobic tail groups comprising a straight or branched hydrocarbon chain, at least a portion of the hydrophobic tail groups having at least one cross-linkable moiety, the aggregates having a polar exterior region comprising the hydrophillic head groups and a non-polar core region comprising said hydrophobic tail groups, the supramolecular complex being stabilized by cross-links formed between the cross-linkable moieties of the hydrophobic tail groups.

Furthermore, methods of making the compositions described above and of using the same in the treatment and diagnosis of various diseases are also within the scope of the present invention. Specifically, in accordance with the present invention, there is provided a method of making supramolecular aggregates of the invention comprising the steps of mixing a predetermined quantity of the above-described phosphonate(s) with a sufficient amount of a biologically acceptable buffer to form a mixture, heating the above-described mixture to boiling, and cooling the above-described mixture, thereby providing an array of aggregates of diverse size, subjecting the resulting mixture to extrusion to produce supramolecular aggregates of uniform size distribution and irradiating the above-described aggregate with a light source generating energy effective to form cross-links between the cross-linkable moieties of the above-described hydrophobic tail groups.

The present invention also provides methods for the treatment of a pathological condition in a patient in need of said treatment, which method comprises administering to the patient at least one of the compositions described herein including a therapeutic agent in an amount having a therapeutic effect on the condition to be treated. Preferably the method is employed to treat a patient with cancer, especially, bone cancer.

The present invention further provides methods for the diagnosis of pathological tissue in a patient in need of said diagnosis. Specifically, the method comprises delivering a diagnostically effective amount of the diagnostic agent-containing compositions of the invention to the pathological tissue site.

### BRIEF DESCRIPTION OF THE DRAWINQS

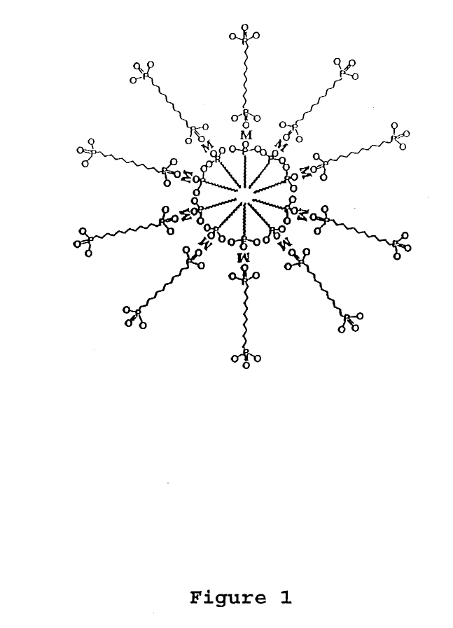

FIG. 1 shows an illustration of one embodiment of the drug delivery vehicle of the invention.

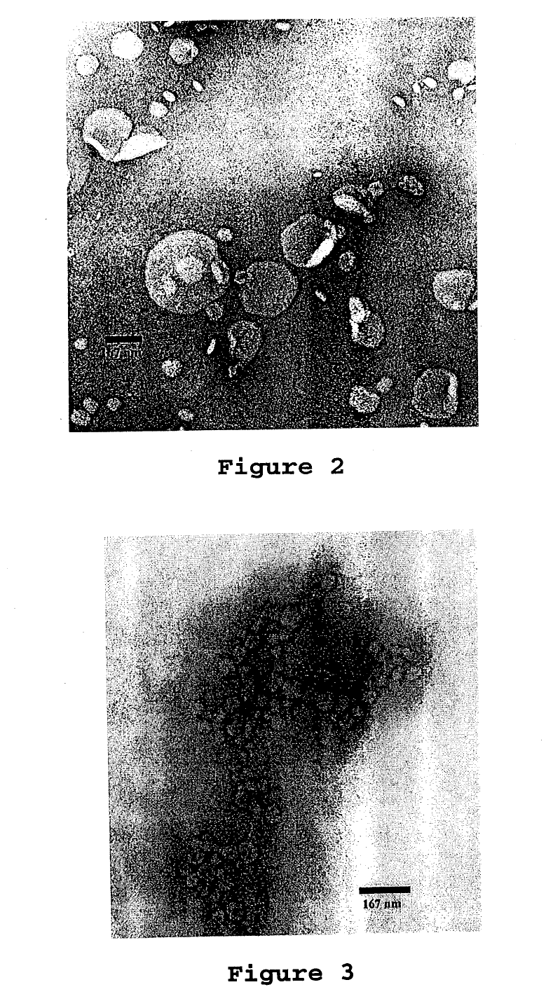

FIG. 2 is a micrograph obtained by transmission electron microscopy (TEM) of a negatively stained image of vesicles prepared using dodeca-5,7-diynoyl phosphonic acid.

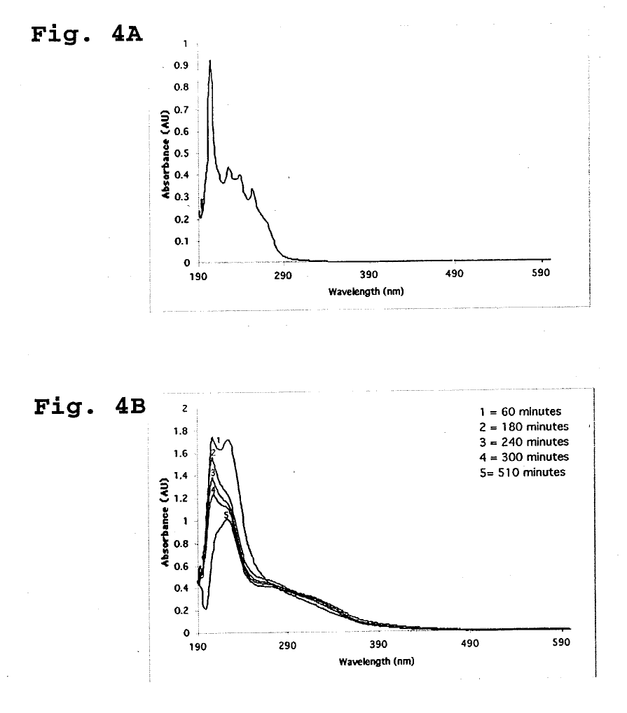

FIG. 3 is a TEM image of vesicles prepared using dodeca-5,7-diynoyl phosphonic acid, one week following initial preparation.

FIG. 4 is a graphical representation of the UV-vis spectrum for vesicles prepared using dodeca-5,7-diynoyl phosphonic acid, in the unpolymerized (a) and polymerized (b) state.

### DETAILED DESCRIPTION OF THE INVENTION

The supramolecular aggregate of the invention may comprise various phosphonate compounds, which are derivatives of phosphonic acid having the formula R—PO(OH)₂, R represents a straight or branched chain hydrocarbon, which has at least one cross-linkable moiety, e.g. unsaturation bonds, and which may have a hydrocarbon chain length of C₆ to C₂₂. In a preferred embodiment the hydrocarbon chain length is between C₈-C₁₆. Alkylphosphronic acids derivatives of the foregoing formula can be prepared according to the following reaction scheme, which is provided by way of example and not limitation.

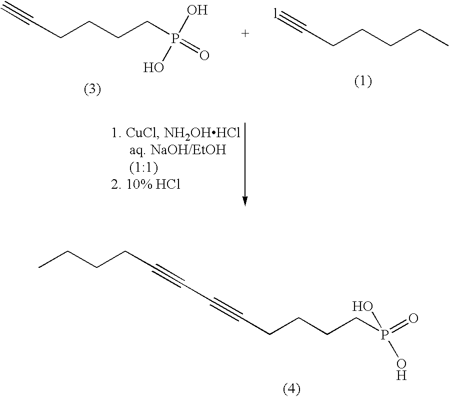

These long chain phosphonates self-assemble into aggregate form when exposed to an aqueous environment under conditions described by Schulz et al. (21).

There are certain advantages in using polymerizable phosphonates to prepare the targeted delivery vehicles of the invention. Chapman and Ringsdorf first used photopolymerizable phospholipids in the early 1980s as a means of preparing aggregates that were stable towards changes in their local environment. (Ringsdorf, H., et al. J. Angew. Chem. (1988) 100: 117); Johnston et al., Biochem. Biophys. Acta (1980) 602: 57. Incorporating the ability to polymerize the aggregates following extrusion enables the size distribution of the vesicles to be “locked-in”. The morphology of the polymerized vesicles should thus remain stable to environmental changes such as pH or metal addition. Moreover, the polymerized aggregates could be directly targeted to bone tissue solely through their terminal phosphonic acid head groups, thus obviating the stabilizing metal layer and the exterior bisphosphonic targeting component, which are included in the “Complexes of Alkylphosphonic Acids”, which is the subject of International Patent Application No. PCT/US00/32057, commonly owned.

The introduction of polymeric character into the vesicle structure can also be used to potentially control the release characteristics of the aggregates. One way this can be accomplished is through variation in the number of unsaturated moieties incorporated into the monomers. By introducing a higher degree of unsaturation, the number of potential cross-linking polymer bonds can be increased and thus the ability of therapeutic or diagnostic agents trapped within this hydrophobic cure region to escape would be reduced.

Another way of controlling the release of active agents is by modifying the hydrophobic tail group of at least one of the phosphonate compounds to include a functional group, which is utilized to covalently bind the therapeutic or diagnostic agent to the aggregate. The covalently bound therapeutic or diagnostic agent may be either hydrophilic or hydrophobic. The chemical nature of the agent will dictate the nature of the functional group(s) utilized for covalent binding thereof. Preferably the bonds thus formed are hydrolyzable. Following polymerization, water molecules near or penetrating the outer region of the hydrocarbon core of the vesicle could slowly degrade these bonds to release the contents of the aggregate. For example, if the therapeutic agent is a compound having a free amine group (—NH₂), the alkylphosphonic acid may be modified to include a free carboxyl group (—COOH), preferably at the terminus of the hydrophobic tail, thereby allowing for covalent binding of the therapeutic agent to the aggregate via an amide bond formed between the amine group and the carboxyl group.

Alternatively, the aggregates may be formed using one or more non-polymerizable phosphonate monomers, thereby to create tiny channels in the aggregates through which trapped therapeutic or diagnostic agent could escape. See, for example, U.S. Pat. No. 5,366,881 to Singh.

In a preferred embodiment of the invention, the composition further comprises a suitable physiologically compatible buffer. Buffers suitable for this purpose are known in the art and include, but are not limited to, phosphate buffered saline (PBS), 3-[N-Morpholino]propanesulfonic acid (MOPS) and 3-[N-Morpholino]ethanesulfonic acid (MES). The compositions of the invention may be administered both systemically as well as injected directly at a disease site.

The means of incorporating therapeutic or diagnostic agents into the aggregates may vary, depending on the relative hydiophobicity of the active agent. Hydrophobic agents can readily be incorporated into the core hydrophobic regions of the aggregate. This provides the most direct and general (i.e. a host of hydrophobic drugs, e.g., paclitaxel, could be used) way to incorporate therapeutic or diagnostic substances into the aggregate delivery system.

Another means of incorporating a drug or diagnostic agent into the aggregates involves the use of a modified monomer phosphonic acid in which the drug is covalently attached to the tail of the amphiphile, as briefly mentioned above. This second approach has been used successfully to attach the hydrophobic anti-cancer drug adriamyacin (ADR) to the interior of several block copolymer micelles (8). For example, the tail of the hydrophobic portion of the alkylphosphonic acid monomer may be converted to a carboxylic acid, which allows for the formation of an amide linkage with the amine on ADR. Upon the breakdown of this linkage, the drug is released from the hydrophobic interior and can diffuse from the aggregate to enter the body. This monomer can be used in various ratios with unmodified phosphonic acid monomers to prepare aggregates.

The supramolecular aggregates described above may have biologically compatible metal ions bound to the exterior thereof in accordance with the present invention. The metal component of the aggregate can optimally be further functionalized with a targeting component for the purpose of enhancing site-specific delivery of the therapeutic or diagnostic agent.

The biologically compatible metal ions are preferably selected from the group consisting of Fe (III), Co (II), Zn (II), Zr (IV), Mg (II) and Ca (II). Of course other biologically compatible metals may be used, if desired. The metal ions are bound to the exterior of the above-described aggregate by direct injection of salt solution into the aggregate preparation, followed by dialysis of excess or unbound ions through dialysis tubing. Solutions were dialyzed for approximately one week.

The targeting component of the composition may include but is not limited to antibody, fragment of an antibody, protein ligand, polysaccharide, polynucleotide, polypeptide, low molecular mass organic molecule and the like. Such targeting group can be linked covalently to the phosphonate, or can be non-covalently incorporated in the compositions, e.g., through hydrophobic, electrostatic interactions or hydrogen bonds.

Preferably the targeting component is α, ω bisphosphonic acid, such as decyl(bis)phosphonic acid (DBPA), geminal bisphosphonic acid (the latter being characterized by a P—C—P bond) or tetrakisphosphonic acid (the latter containing two P—C—P bisphosphonate moieties connected by a carbon chain). Because DBPA is soluble in water, the addition of this component to the aggregates can be carried out at room temperature (i.e. similar conditions to those used for preparing self-assembled multilayers with DBPA) (20). Size and molecular weight of the resulting delivery vehicle may be determined by static and dynamic light scattering and TEM acquired by negatively staining the samples. Due to the large negatively charged acid head groups on the exterior of the resulting delivery vehicle system, it should have a long circulation time within the bloodstream and not be taken up by other organs in the body (16, 17).

In order to enhance release from these metallated materials, optionally, one may add small amounts of impurities, such as chemically distinct surfactant, e.g., one with a sulfonate head group, which is unable to bind metal ions. This would reduce the number of sites available for metal binding on the exterior of the aggregate and should increase the fluidity of the outer shell and promote release. Suitable surfactants for this purpose are described in Liposomes: A Practical Approach, IRL Press, Oxford, 1997.

The targeting component may be chemically modified to allow for binding of the therapeutic or diagnostic agent thereto.

The diagnostic agents that may be used in the practice of this invention include those having utility in imaging. Suitable therapeutic agents are those capable of acting on a cell, organ or organism to create a change in the functioning of the cell, organ or organism, including but not limited to pharmaceutical agents or drugs. Such agents include a wide variety of substances that are used in therapy, immunization or otherwise are applied to combat human and animal disease. Such agents include but are not limited to analgesic agents, anti-inflamatory agents, antibacterial agents, antiviral agents, antifungal agents, antiparasitic agents, tumoricidal or anti-cancer agents, proteins, toxins, enzymes, hormones, neurotransmitters, glycoproteins, immunoglobulins, immunomodulators, dyes, radiolabels, radio-opaque compounds, fluorescent compounds, polysaccharides, cell receptor binding molecules, anti-inflammatories, anti-glaucomic agents, mydriatic compounds and local anesthetics.

Exemplary non-steroidal anti-inflamatories include, but are not limited to, indomethacin, salicylic acid acetate, ibuprofen, sulindac, piroxicam, and naproxen, antiglaucomic agents such as timolol or pilocarpine, neurotransmitters such as acetylcholine, anesthetics such as dibucaine, neuroleptics such as the phenothiazines (e.g., compazine, thorazine, promazine, chlorpromazine, acepromazine, aminopromazine, perazine, prochlorperazine, trifluoperazine, and thioproperazine), rauwolfia alkaloids (e.g., resperine and deserpine), thioxanthenes (e.g., chlorprothixene and tiotixene), butyrophenones (e.g., haloperidol, moperone, trifluoperidol, timiperone, and droperidol), diphenylbutylpiperidines (e.g., pimozde), and benzamides (e.g., sulpiride and tiapride); tranquilizers such as glycerol derivatives (e.g., mephenesin and methocarbamol), propanediols (e.g., meprobamate), diphenylmethane derivatives (e.g., orphenadrine, benzotrapine, and hydroxyzine), and benzodiazepines (e.g., chlordiazepoxide and diazepam); hypnotics (e.g., zolpdem and butoctamide); beta-blockers (e.g., propranolol, acebutonol, metoprolol, and pindolol); antidepressants such as dibenzazepines (e.g., imipramine), dibenzocycloheptenes (e.g., amtiriptyline), and the tetracyclics (e.g., mianserine); MAO inhibitors (e.g., phenelzine, iproniazid, and selegeline); psychostimulants such as phenylehtylamine derivatives (e.g., amphetamines, dexamphetamines, fenproporex, phentermine, amfeprramone, and pemoline) and dimethylaminoethanols (e.g., clofenciclan, cyprodenate, aminorex, and mazindol); GABA-mimetics (e.g., progabide); alkaloids (e.g., codergocrine, dihydroergocristine, and vincamine); anti-Parkinsonism agents (e.g., L-dopamine and selegeline); agents utilized in the treatment of Altzheimer's disease, cholinergics (e.g., citicoline and physostigmine); vasodilators (e.g., pentoxifyline); and cerebro active agents (e.g., pyritinol and meclofenoxate).

Anti-neoplastic agents can also be used advantageously as biological agents in the compositions of the invention. Representative examples include, but are not limited to paclitaxel, daunorubicin, doxorubicin, carminomycin, 4′-epiadriamycin, 4-demethoxy-daunomycin, 11-deoxydaunorubicin, 13-deoxydaunorubicin, adriamycin-14-benzoate, adriamycin-14-actanoate, adriamycin-14-naphthaleneacetate, vinblastine, vincristine, mitomycin C, N-methyl mitomycin C, bleomycin A₂, dideazatetrahydrofolic acid, aminopterin, methotrexate, cholchicine and cisplatin. Representative antibacterial agents are the aminoglycosides including gentamicin. Representative antiviral compounds are rifampicin, 3′-azido-3′-deoxythymidine (AZT), and acylovir. Representative antifungal agents are the azoles, including fluconazole, macrolides such as amphotericin B, and candicidin. Representative anti-parastic compounds are the antimonials. Suitable biological agents also include, without limitation vinca alkaloids, such as vincristine and vinblastine, mitomycin-type antibiotics, such as mitomycin C and N-methyl mitomycin, bleomycin-type antibiotics such as bleomycin A₂, antifolates such as methotrexate, aminopterin, and dideaza-tetrahydrofolic acid, taxanes, anthracycline antibiotics and others.

The compositions of this invention also can utilize a variety of polypeptides, such as antibodies, toxins, such as diphtheria toxin, peptide hormones, such as colony stimulating factor, and tumor necrosis factors, neuropeptides, growth hormone, erythropoietin, and thyroid hormone, lipoproteins such as μ-lipoprotein, proteoglycans such as hyaluronic acid, glycoproteins such as gonadotropin hormone, immunomodulators or cytokines such as the interferons or interleukins, as well as hormone receptors such as the estrogen receptor.

The compositions also can comprise enzyme inhibiting agents such as reverse transcriptase inhibitors, protease inhibitors, angiotensin converting enzymes, 5μ-reductase, and the like. Typical of these agents are peptide and nonpeptide structures such as finasteride, quinapril, ramipril, lisinopril, saquinavir, ritonavir, indinavir, nelfinavir, zidovudine, zalcitabine, allophenylnorstatine, kynostatin, delaviridine, bis-tetrahydrofuran ligands (see, for example Ghosh et al., J. Med. Chem. 1996, 39: 3278), and didanosine. Such agents can be administered alone or in combination therapy; e.g., a combination therapy utilizing saquinavir, zalcitabine, and didanosine, zalcitabine, and zidovudine. See, for example, Collier et al., Antiviral Res. 1996, 29: 99.

The compositions described herein may also comprise nucleotides, such as thymine, nucleic acids, such as DNA or RNA, or synthetic oligonucleotides, which may be derivatized by covalently modifying the 5′ or the 3′ end of the polynucleic acid molecule with hydrophobic substituents to facilitate entry into cells (see for example, Kabanov et al., FEBS Lett. 1990, 259, 327; Kabanov and Alakhov, J. Contr. Rel. 1990, 28: 15). Additionally, the phosphate backbone of the polynucleotides may be modified to remove the negative charge (see, for example, Agris et al., Biochemistry 1968, 25:6268, Cazenave and Helene in Antisense Nucleic Acids and Proteins: Fundamentals and Applications, Mol and Van der Krol, Eds., p. 47 et seq., Marcel Dekker, New York, 1991), or the purine or pyrimidine bases may been modified, for example, to incorporate photo-induced crosslinking groups, alkylating groups, organometallic groups, intercalating groups, biotin, fluorescent and radioactive groups (see, for example, Antisense Nucleic Acids and Proteins: Fundamentals and Applications, Mol and Van der Krol, Eds., p. 47 et seq., Marcel Dekker, New York, 1991; Milligan et al., In Gene Therapy for Neoplastic Diseases, Huber and Laso, Eds. P. 228 et seq., New York Academy of Sciences, New York, 1994). Such nucleic acid molecules can be, among other things, antisense nucleic acid molecules, phosphodiester, oligonucleotide α-anomers, ethylphospotriester analogs, phosphorothioates, phosphorodithioates, phosphoroethyletriesters, methylphosphonates, and the like (see, e.g., Crooke, Anti-Cancer Drug Design 1991, 6: 609; De Mesmaeker et al, Acc. Chem. Res. 1995, 28: 366).

The compositions of the invention may also include antigene, ribozyme and aptamer nucleic acid drugs (see, for example, Stull and Szoka, Pharm. Res. 1995, 12: 465).

Other suitable biologically active agents include oxygen transporters (e.g. porphines, porphirines and their complexes with metal ions), coenzymes and vitamins (e.g. NAD/NADH, vitamins B12, chlorophylls), and the like.

Suitable biologically active agents further include those used in diagnostic visualization methods, such as magnetic resonance imaging (e.g., gadolinium (III) diethylenetriamine pentaacetic acid), and may be a chelating group (e.g., diethylenetriamine pentaacetic acid, triethylenetriamine pentaacetic acid, ethylenediamine-tetraacetic acid, 1,2-diaminocyclo-hexane-N,N,N′,N′-tetraaceticacid, N,N′-di (2-hydroxybenzyl) ethylene diamine), N-(2-hydroxyethyl) ethylene diamine triacetic acid and the like). Such agents may further include an alpha-, beta-, or gamma-emitting radionuclide (e.g., galliun 67, indium 111, technetium 99). Iodine-containing radiopaque molecules are also suitable diagnostic agents. The diagnostic agent may also include a paramagnetic or superparamagnetic element, or combination of paramagnetic element and radionuclide. The paramagnetic elements include but are not limited to gadolinium (III), dysporsium (III), holmium (III) europium (III) iron (III) or manganese (II).

The compositions of the present invention allow diverse routes of administration, including but not limited to parenteral (such as intramuscular, subcutaneous, intraperitoneal, and intraveneous), oral, otic, topical, vaginal, pulmonary, and ocular. These compositions can take the form of aqueous solutions, suspensions, micelles, vesicles, emulsions and microemulsions.

Conventional pharmaceutical formulations may be employed. When aqueous suspensions are required for oral use, the composition can be combined with emulsifying and suspending agents. For parenteral administration, sterile solutions of the composition are usually prepared, and the pH of the solutions are suitably adjusted and buffered. For intravenous use, the total concentration of solutes should be controlled to render the preparation isotonic. For ocular administration, ointments or droppable liquids may be delivered by well-known ocular delivery systems such as applicators or eyedroppers. Such compositions can include mucomimetics such as hyaluronic acid, chondroitin sulfate, hydroxypropyl methylcellulose or polyvinyl alcohol, preservatives such as sorbic acid, EDTA or benzylchronium chloride, and the usual quantities of diluents and/or carriers. For pulmonary administration, diluents and/or carriers will be selected to be appropriate to allow for formation of an aerosol.

It is known that phosphonic acid moieties bind specifically to bone tissue. For about the past two decades geminal bisphosphonic acids have been increasingly studied for use as therapeutic agents for osteoporosis (16,17). Osteoporosis is a condition in which the natural process of bone breakdown by osteoclastic cells (resorption) is dramatically increased without subsequent reformation of new tissue (16). Rapid breakdown of the bone matrix dramatically increases the amount of exposed Ca++ at the bone surface, and the strongly binding phosphonic acids are readily targeted to the affected area. Although the exact mechanism is still unknown, it is believed that the negatively charged acid headgroups bind tightly to exposed calcium ions thus blocking osteoclastic activity (15,17). This binding “locks” the calcium in place strengthening the weakened tissue, while providing a protective coat over the surface to prevent further resorption (15,17). The compositions of the invention are, therefore, unique in that they enable targeted delivery of an encapsulated therapeutic or diagnostic agent to one specific site.

When a cancer cell metastasizes to the bone, it also triggers the release of chemical stimulators which increase bone breakdown (15). Using the knowledge that phosphonic acids will specifically target areas where resorption is occurring rapidly, a phosphonic acid derivative is used to target drug delivery to areas where cancer has metastasized. The resulting functionalized aggregate has free acids extended out from the main delivery vehicle assembly which serve to target drug delivery.

The following examples are provided to illustrate certain embodiments of the present invention. These examples set forth the best mode presently contemplated for carrying out the invention described herein; they are not intended to limit the invention in any way.

### EXAMPLE 1

The materials used in this example, including 1-chloro-5-hexyne,1-hexyne, copper(I)chloride, hydroxylamine hydrochloride, bromotrimethylsilane, butyl lithium (2.5 M in hexanes), reagent grade sodium hydroxide, and ethyl amine were used as received from Aldrich Chemical company. Triethylphosphite (98%) was purchased from Aldrich Chemical Company and distilled from sodium metal under nitrogen prior to use. Diethyl ether was purchased from Fisher Chemical and was distilled from sodium prior to use. ¹H and ¹³C NMR spectra were acquired using a Varian Unity-INOVA 300 spectrometer at room temperature. Infrared spectra were recorded using a Nicolet Magna 550 FT-IR with Omnic software. UV-vis spectra were acquired on a HP 8453 UV-vis Spectrometer attached to a HP Kayak XA running ChemStation (Rev A.06.01) software. The melting points of the compounds were determined using a Mel-Temp II capillary melting point apparatus equipped with a thermocouple. Differential scanning calorimetry experiments were conducted using a TA Instruments 2920 Modulated DSC.

1-Iodo-1-hexyne (1). Ether (30 ml) was transferred via cannula to a clean, flame dried, 500 ml 3 neck round bottom flask with stir bar that was fitted with a rubber septum, a glass stopper, and an equalizing addition funnel. 1-Hexyne (10.04 g, 0.121 moles) was added to the flask and stirred while being cooled in a dry ice acetone bath to −50° C. After reaching temperature, 2.5 M butyl lithium (46 ml, 0.115 moles) was added dropwise to the stirring reaction mixture. Immediately following the addition of this solution, iodine (20.36 g 0.16 moles) dissolved in ether (60 ml) was added dropwise through the addition funnel. Following the addition of this solution the reaction was transferred to an ice bath and brought to OC. The reaction was quenched through the slow addition of water to the reaction mixture with vigorous stirring. The biphasic mixture was transferred to a separation funnel and the water layer was removed. The ether layer was washed two more times with water discarding the water layer after each wash. The ether layer was washed once with an aqueous sodium thiosulfate solution followed by one more water wash. The ether layer was then dried over sodium sulfate and the solvent removed using rotary evaporation. No further purification was attempted as ¹H NMR showed only a slight trace of starting material. 16.56 g (66%) of I was isolated as an orange oil. ¹H NMR (CDCl³): δ 0.91 (t, 3H), 1.46 (m, 4H), and 2.37 (t, 2H).

1-Iodo-5-hexyne (2). Sodium iodide (50.1 g,0.88 moles) and 1-chloro-5-hexyne (13.13 g, 0.111 moles) was dissolved in 250 ml of reagent grade acetone and the reaction mixture refluxed for 12 hours. The reaction was cooled and the white precipitate removed using filtration. The acetone solution was reduced in volume using rotary evaporation and 100 ml of water was added. This solution was extracted with ether and the resulting ether layer washed three times with water before drying over sodium sulfate. The ether was removed under vacuum. The resulting product was distilled (55° C. @ 2 mm Hg) to yield 20.92 g (90%) of II as a yellow oil. ¹H NMR (CDCl₃): δ1.65 (q, 2H), 1.96 (m, 3H), 2.24 (td, 2H), and 3.22 (t, 2H). IR: 3295, 2940, 2861, 2836, 2117, 1430, and 1211 cm−1.

1-Hexynyl phosphonic acid (3) In a three neck flask equipped with a stir bar and set up for simple distillation, molecule 2 (9.13 g, 0.044 moles) was added. Into this triethylphosphite (7.65 g, 0.066 moles) was cannulated under nitrogen and the reaction was heated to 140° C. for 4 hours. The reaction was then cooled to room temperature and distilled under vacuum (94° C. @2 mm Hg) to collect 4.13 g (43%) of a colorless oil. Following distillation, 3.14 g (0.0144 moles) of the oil was transferred to a 50 ml round bottom flask fitted with a small stir bar and rubber septum along with 20 ml of dichloromethane. The mixture was stirred to ensure complete dissolution of the phosphonate before bromotrimethylsilane (3.79 ml, 0.0248 moles) of was added dropwise via gas tight syringe. The reaction was allowed to stir over a period of 12 hours. 3 M aqueous sodium hydroxide was added slowly until the pH of the solution was 10 in order to quench the reaction. This reaction mixture was extracted into chloroform three times. The chloroform was dried briefly over sodium sulfate before being removed by rotary evaporation to yield an off white crystalline product. The water layers from the earlier extraction were also evaporated to dryness and the remaining solid dissolved in acetone. Filtration of this solution to remove sodium chloride also yielded a tiny amount of product that was combined with that from the chloroform extraction. Recrystallization of this material from a 10:90 ethyl acetate:petroleum ether solvent mixture yielded 0.095 g (4.1%) of white crystals that were pure by 1H NMR spectroscopy. Melting point 114.4-115.7° C. ¹H NMR (CDCl₃): δ1.77 (m, 6H), 1.97 (t, 2H), 2.23 (td, 2H), and 6.04 (s, 2H). ³¹P NMR (CDCl₃) δ 37.180. IR: 3290, 2946, 2117, 1450, 1292, 1101, and 995 cm⁻¹.

Dodeca-5,7-diynoyl phosphonic acid (4). In a clean 3 neck round bottom flask with stir bar and fitted with a rubber septum, an equalizing addition funnel, and a glass stopper, sodium hydroxide (0.026 g, 6.48×10⁻⁴ moles) was dissolved in ethanol/water (3 ml, 1:1). To this solution 3 (0.095 g, 5.89×10-4 moles) was added and stirred to dissolve. In a separate flask ethylamine (0.5 ml, 30% aqueous) was combined with copper(I)chloride (0.014 g, 1.47×10⁻⁴ moles). Following the complete dissolution of the solid copper, this mixture was transferred via pipette to the stirring phosphonic acid mixture. To this deep blue solution a few crystals of hydroxylamine hydrochloride were added until the reaction turned a yellow green color. To the stirring reaction mixture 1 (0.122 g, 5×10⁻⁴ moles) was added dropwise from a gas tight syringe. Aqueous hydroxylamine hydrochloride (10%) was added through the addition funnel as needed to keep the copper catalyst active (indicated by the yellow green color of the reaction). Following the addition of 1, the solution was allowed to stir for 1 hour before 10% HCl was added to quench the reaction. The mixture was transferred to a separatory funnel and extracted three times with ether. The ether was dried over sodium sulfate and dried using rotary evaporation to yield white crystals. The crystals were recrystallized from ethyl acetate/petroleum ether (10:90) to yield 0.023 g (14.5%) of white crystals that were pure by NMR, product 4. Melting point 135.4-137.0° C. ¹H NMR (CDCl₃): δ 0.91 (t, 3H), 1.46 (m, 6H), 1.71 (m, 4H), 2.28 (m, 4H), and 7.52 (s, 2H) ³¹P NMR (CDCl₃): δ 37.05. Mass Spectrometry (M⁺) 243.1 Hz.

Twice the value of the CMC determined for n-dodecanephosphonic acid (2.94×10⁻⁴ M)⁴ was used to assure aggregate formation in these experiments due to the limited amount of the final product. All samples were prepared in the manner described by Schulz, supra, using solutions buffered to a pH of 5.5 with 5 mM MES.

Samples prepared in the above manner were imaged using TEM by negatively staining the samples with 1% uranyl acetate. FIG. 2 is a picture of unpolymerized vesicles of dodeca-5,7-diynoyl phosphonic acid within four days of their initial preparation. It appears from this micrograph that the aggregates are small and relatively spherical compared to the fully saturated aggregates of n-dodecanephosphonic acid prepared as described hereinbelow. The average size of these vesicles is roughly 50 nm in diameter with the sizes of individual aggregates ranging from 4 nm to over 400 nm. Interestingly, it appears that some of these vesicles tend to roll into cylinders. This behavior was only observed with freshly prepared aggregates prior to their exposure to light. One explanation for this behavior is that it represents an artifact of drying the sample in the presence of the metal stain used to visualize it.

FIG. 3 is a micrograph of the same sample after one week. It is noteworthy that the vesicles are still spherical though they now appear to be smaller and have a more irregular exterior surface. The average size of the aggregates is still close to 50 nm in diameter, but the size dispersity has dramatically decreased with no evidence of vesicles larger than 100 nm present in the sample. Moreover, there was no evidence of the rolled cylinders previously imaged in the fresh aggregate solutions.

A fresh solution of the vesicles of dodeca-5,7-diynoyl phosphonic acid was prepared and aged three days before polymerization using an immersion UV light operating at 450 W. The polymerization of the aggregates was followed using UV-vis spectroscopy and the results are shown in FIG. 4. FIG. 4A is the spectrum of the unpolymerized vesicle. As the polymerization proceeds there is a red shift in the spectrum as the conjugation of the system increases. See FIG. 4B. Within the first hour of polymerization the peaks at 239 nm and 254 nm completely disappear while the peak centered at 226 nm grows dramatically. This peak slowly coalesces with the peak at 209 nm to form a broad featureless peak after 510 minutes of polymerization. Extending the polymerization time past this point produced no further changes in the UV-vis spectrum of this sample.

The dodeca-5,7-diynoyl phosphonic acid obtained in this example readily dissolves in buffered aqueous solutions to form vesicles. Using TEM to analyze these phosphonic acid vesicles it appears that the age of the solution affects the size dispersity and possibly the stability of the aggregates. Aggregates that had aged one week appeared to be smaller and more monodipserse compared to solutions less than five days old. This may be due to polymerization of small amounts of the phosphonic acid in the aggregate solutions upon exposure to ambient light.

### EXAMPLE 2

An aggregate was prepared using n-dodecanephosphonic acid. Specifically, a 3×10⁻⁴ molar (on average) solution of n-dodecanephosphonic acid was used, which was prepared by adding 17 mg dodecanephosphonic acid to 250 mL of a 5 mM solution of morpholinoethanesulfonic acid (MES) buffer solution. This solution was heated in a boiling water bath for 40-60 minutes. The solution was then cooled and allowed to sit undisturbed for 3 days (following Shulz' procedure (21)). This preparation was used to produce the desired micelles and the reported critical micelle concentration (CMC) was independently confirmed through measurement of the surface tension by means of a Wilhelmy Plate Balance (22). It was found that the aggregates formed through this preparation are sperical in shape with an average diameter of 20-120 nm as visualized through TEM. It has also been determined that these aggregates are stable over the pH range of 4-6, with minimal structural changes.

The choice of biologically compatible metal ion used to bind to the micelle exterior directly influences the size and the shape of the resulting aggregates. Iron (III) salts are preferred because they are relatively inexpensive, readily available in several simple forms and known to be biologically compatible. However, other metal ions may be used if desired, including, without limitation, Zr (IV), Mg(II), Ca(II), Co(II) and Zn(II) salts. The TEM images in FIG. 3 represent three aggregate samples from the same starting micelle solution that have been metallated with different iron salt sources; FIG. 3A: Fe(ClO₄)₃; FIG. 3B: Fe(NO₃)₃; FIG. 3C: FeCl₃.

The aggregates in each of these samples are similar, ranging in size from 25 nm to 275 nm in diameter with an average size of 110 nm. Aggregates of a relatively uniform size distribution can be obtained by multiple extrusions through a Nuclepore filter® (Whatman) prior to metallation. Specifically this entails multiple extrusions through a membrane filter to yeild aggregates that are more monodisperse in size, i.e., on the order of 25-75 nm.

The diversity of sizes of the metallated aggregates and the change in shape relative to the metal-free aggregates can only be explained if some type of reorganization is occurring upon addition of the metal. For the large spheres observed upon metallation with Fe(ClO₄)₃₁ the size of these aggregates indicates reorganization to form larger vesicular species. This type of reorganization phenomenon has been previously reported in vesicle systems with the addition of group II elements such as magnesium and calcium (23,24). In these systems it was found that the addition of metal ions to the exterior of the vesicle negated the surface charge of the exposed headgroups. This in turn allowed the aggregates to approach each other closely and fuse together to form larger more stable “super vesicles” (23,24).

### REFERENCES

1) “The Challenge of Liposome Targeting In Vivo” Poste, G.; Kirsch, R.; Koestler, T. Liposome Technology, Vol.3, Ed: Gregory Gregoriadis, CRC Press, Boca Raton (1984), p.1.

2) Antitumor Drug-Saccharaide Conjugate Exhibiting Cell-Specific Antitumor Activity,” Ouchi, T.; Kobayashi, H.; Hirai, K.; Ohya, Y. in Polymeric Delivery Systems, Eds: M. A. El-Nokoly, D. M. Piatt, B. A. Charpentier, ACS Press (1993), p. 382.

3) Allen, C., Yu, Y., Maysinger, D, Eisenberg, A. Bioconjugate Chem. 1998, 9, 564-572.

4) El-Nokaly, M. A.; Piatt, D. M.; Charpentier, B. A. Polymeric Delivery Systems; American Chemical Society: Washington, D.C., 1993, Vol 520.

5) “Theoretical and Practical Considerations in Preparing Liposomes for the Purpose of Releasing Drug in Response to Changes in Temperature and pH,” Yatvin, M. B.; Cree, T. C.; Tegmo-Larsson, I. -M., Liposome Technology, Vol.2, Ed: Gregory Gregoriadis, CRC Press, Boca Raton (1984), p.157.14) Allen, T. M. Trands Pharmacol. Sci., 15, 215.

6) “Preparation and Characterization of Self-Assembled Polymer-Metal Complex Micelle from cis-Dichlorodiammineplatinium(II) and Poly(ethyleneglycol)-Poly((((-aspartic acid) Block Copolymer in an Aqueous Medium,” Nishiyama, N.; Yokoyama, M.; Aoyagi, T.; Okano, T.; Sakurai, Y.; Kataoka, K. Langmuir 1999, 15, 377.

7) “Preparation of Micelle Forming Polymer Drug Conjugates,” Yokoyama, M.; Kwon, G. S.; Okano, T.; Sakurai, Y.; Seto, T.; Kataoka, K. Bioconj. Chem. 1992, 3, 295.

8) “Biodegradable Block Copolymers as Injectable Drug-Delivery Systems,” Jeong, B.; Bae, Y. H.; Lee, D. S.; Kim, S. W. Nature 1997, 388, 860.

9) “Toxicity and Antitumor Activity Against Solid Tumors of Micelle-forming Polymeric Anticancer Drug and Its Extremely Long Circulation in the Blood,” Yokoyama, M.; Okano, T.; Sakurai, Y.; Ekimoto, H.; Shibazaki, C.; Kataoka, K. Cancer Res. 1991, 51, 3229.

10) “New Methods of Drug Delivery,” Langer, R.; Science 1990, 249, 1527.

11) Fendler, J. H.; Fendler, E. J. Catalysis in Micellar and Macromolecular Systems; Academic Press: College Station, Tex., 1975.

12) Fendler, J. H. Membrane Mimetic Chemistry; John Wiley and Sons: New York, 1982.

13) “Lipid Polymorphism and the Functional Roles of Lipids in Biological Membranes,” Cullis, P. R.; De Kruijff, B. Biochim. Biophysica Acta 1979, 559, 399.

14) “Bisphosphonates Spearhead New Approach to Treating Bone Metastases,” Bankhead, C; J. Nat. Cancer Inst. 1997, 89, 115.

15) Fleisch, H. Bisphosphonates: Mechanisms of Action and Clinical Use; Fleisch, H., Ed.; Springer-Verlag: New York, 1993, pp 377.

16) “Bisphosphonates: Mechanisms of Action,” Rodan, G. A.; Fleisch, H. A. J. Clinical Investigation 1996, 97, 2692.

17) “Growth and Characterization of Metal (II) Alkanebisphosphonate Multilayer Thin Films on Gold Surfaces,” Yang, H. C.; Aoki, K.; Hong, H. -G.; Sackett, D. D.; Arendt, M. F.; Yau, S. -L; Bell, C. M.; Mallouk, T. E. J. Am Chem. Soc. 1993, 115, 11855.

18) “Layered Metal Phosphates and Phosphonates: from Crystals to Monolayers,” Cao, G.; Hong, H. -G.; Mallouk, T. E. Acc. Chem. Res. 1992, 25, 420

19) “Variation of Layer Spacing in Self-Assembled Hafnium 1,10-decanediylbis(phosphonate) Multilayers as Determined by Ellipsometry and Grazing Angle X-ray Diffraction,” Zeppenfeld, A. C.; Fiddler, S. L.; Ham, W. K.; Klopfenstein, B. J.; Page, C. J. J. Am. Chem. Soc. 1994, 116, 9158.

20) “FTIR Studies of Hafnium-(,(-Alkylbisphosphonate Multilayers on Gold: Effects of Bisphosphonate Chain Length, Substrate Roughness, and Functionalization on Film Structure and Order” O'Brien, J. T.; Zeppenfield, A. C.; Richmond, G. L.; Page, C. J. Langmuir 1994, 10, 4657.

21) “The Aggregation of n-Dodecanephosphonic Acid in Water” Minardi, R. M.; Schulz, P. C.; Vuano, B. Colloid Polym Sci. 1996, 274, 1089.

22) Becher, P. Emulsions: Theory and Practice; Reinhold Publishing: New York, 1965.

23) “Lipid Vesicles as Carriers for Introducing Materials into Cultured Cells: Influence of Vesicle Lipid Concentration on Mechanism of Vesicle Incorperation into Cells” Poste, G.; Papahadjopoulos, D. Proc. Natl. Acad. Sci. U.S.A. 1976, 73, 1603.

24) Papahadjopoulos, D.; Vail, W. J.; Newton, C.; Nir, S.; Jacobson, K.; Poste, C.; Lazo, R. Biochim. Biophys. Acta 1977, 465, 579.

25) “Formation of Unilamellar Vesicles” Lasic, D. D. J. Colliod Interface Sci. 1988, 124, 428.

26) “Characterization of Vesicles by Classical Light Scattering” Van Zanten, J. H.; Monbouquette, H. G. J. Colloid Inter. Sci. 1991, 146, 330.

While certain preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A composition which comprises a therapeutic or diagnostic agent and stabilized supramolecular aggregates comprising a phosphonate or a combination of chemically distinct phosphonates, said phosphonate(s) having hydrophillic head groups, and hydrophobic tail groups comprising a straight or branched hydrocarbon chain, at least a portion of said hydrophobic tail groups having at least one cross-linkable moiety, said aggregates having a polar exterior region comprising said hydrophillic head groups and a non-polar core region comprising said hydrophobic tail groups, said supramolecular aggregates being stabilized by cross-links formed between the cross-linkable moieties of said hydrophobic tail groups.

2. A composition as claimed in claim 1, wherein said phosphonate comprises a long chain alkyl phosphonic acid.

3. A composition as claimed in claim 1, wherein said at least one cross-linkable moiety comprises at least one unsaturated bond in the hydrocarbon chain of said hydrocarbon tail group.

4. A composition as claimed in claim 3, wherein said unsaturated bond is a triple bond.

5. A composition as claimed in claim 1 in the form of micelles.

6. A composition as claimed in claim 1 in the form of vesicles.

7. A composition as claimed in claim 1, which includes a therapeutic agent.

8. A composition as claimed in claim 7, wherein said therapeutic agent is hydrophobic and is incorporated in the non-polar core region of said supramolecular aggregate.

9. A composition as claimed in claim 7, wherein the hydrophobic tail group of at least one of said phosphonates is modified to include a functional group, and said therapeutic agent is covalently bound to said supramolecular aggregate through said functional group.

10. A composition as claimed in claim 9, wherein said therapeutic agent is hydrophobic.

11. A composition as claimed in claim 10, wherein said therapeutic agent is a compound having a free amine group (—NH₂), said at least one phosphonate is modified to include a free carboxyl group (—COOH) and said therapeutic agent is covalently bound to said aggregate by an amide bond formed between said amine group and said carboxyl group.

12. A composition as claimed in claim 1, wherein said supramolecular aggregate further comprises at least one biologically compatible metal ion bound to said polar exterior region and, optionally, a targeting component bound to said metal ion(s), said targeting component being effective to deliver said composition to a pathologic site in vivo.

13. A composition as claimed in claim 12, wherein said supramolecular aggregate comprises dodeca-5,7-diynoyl phosphonic acid, said biologically compatible metal ions are selected from the group consisting of Fe (III), Zr (IV), Mg (II), Ca (II), Co (II) and Zn (II), and said therapeutic agent is adriamycin.

14. A composition as claimed in claim 12, which further includes said targeting component, which is selected from the group consisting of α, ω, bisphosphonic acid and a geminal bisphosphonic acid.

15. A composition as claimed in claim 12, wherein said supramolecular aggregate further includes a surfactant that does not bind to metal ions.

16. A composition as claimed in claim 15, wherein said surfactant has a sulphonate head group.

17. A drug delivery composition which comprises multiple micelles comprising the composition of claim 7 in a physiologically compatible carrier medium.

18. A drug delivery composition which comprises multiple vesicles comprising the composition of claim 7 in a physiologically compatible carrier medium.

19. A composition as claimed in claim 1, which includes a diagnostic agent.

20. A composition as claimed in claim 1, wherein said aggregates are of uniform size.

21. A method of making supramolecular aggregates comprising a phosphonate or a combination of chemically distinct phosphonates, said phosphonate(s), having hydrophilic head groups and hydrophobic tail groups comprising a straight or branched hydrocarbon chain, at least a portion of said hydrophobic tail groups having at least one cross-linkable moiety, said aggregates having a polar exterior region comprising said hydrophilic head groups and a non-polar core region comprising said hydrophobic tail groups, said method comprising:
a. mixing a predetermined quantity of said phosphonate(s) with a sufficient amount of a biologically acceptable buffer to form a mixture, heating said mixture to boiling, and cooling said mixture, thereby providing an array of aggregates of diverse size;
b. subjecting the resulting mixture to extrusion to produce supramolecular aggregates of uniform size distribution; and
c. irradiating said aggregate with a light source generating energy effective to form cross-links between the cross-linkable moieties of said hydrophobic tail groups.

22. A method as claimed in claim 21, wherein said cross-linkable moiety comprises at least one triple bond in the hydrocarbon chain of said hydrophobic tail groups, and said light source generates UV light.

23. A method as claimed in claim 21, wherein said phosphonate comprises dodeca-5,7-diynoyl phosphonic acid.

24. The method of claim 21, wherein said mixture is subjected to repeated extrusions.

25. A method for treatment of a pathological condition in a patient in need of said treatment, said method comprising administering to said patient a composition according to claim 1, wherein the therapeutic agent is present in an amount having a therapeutic effect on the condition to be treated.

26. The method of claim 25, wherein said therapeutic agent is selected from the group consisting of analgesic agents, anti-inflammatory agents, antibacterial agents, antiviral agents, antifungal agents, antiparasitic agents, tumoricidal or anti-cancer agents, proteins, toxins, enzymes, hormones, neurotransmitters, glycoproteins, immunoglobulins, immunomodulators, dyes, radiolabels, radio-opaque compounds, fluorescent compounds, polysaccharides, cell receptor binding molecules, anti-inflammatories, anti-glaucomic agents, mydriatic compounds and local anesthetics.

27. A method of treating cancer in a patient in need of said treatment, said method comprising administering to said patient a therapeutically effective amount of the composition of claim 1, wherein said therapeutic agent is an anti-cancer drug.

28. The method of claim 27, wherein said anti-cancer drug is hydrophobic.

29. The method of claim 27, wherein said anti-cancer drug is selected from the group consisting of paclitaxel, daunorubicin, doxorubicin, carminomycin, 4′-epiadriamycin, 4-demethoxy-daunomycin, 11-deoxydaunorubicin, 13-deoxydaunorubicin, adriamycin-14-benzoate, adriamycin-14-actanoate, adriamycin-14-naphthaleneacetate, vinblastine, vincristine, mitomycin C, N-methyl mitomycin C, bleomycin A₂, dideazatetrahydrofolic acid, aminopterin, methotrexate, cholchicine and cisplatin.

30. The method of claim 27, wherein said cancer is bone cancer.
